(19) United States
(12) Patent Application Publication
(10) Pub. No.: US 2021/0361700 A1
(43) Pub. Date: Nov. 25, 2021

(54) NUTRITIONAL SUPPLEMENT TO COMBAT COVID-19 AND AID VACCINATION

(71) Applicant: Trustees of Tufts College, Medford, MA (US)

(72) Inventors: Simin Nikbin Meydani, Newton, MA (US); Dariush Mozaffarian, Waban, MA (US)

(21) Appl. No.: 17/326,830

(22) Filed: May 21, 2021

Related U.S. Application Data
(60) Provisional application No. 63/029,157, filed on May 22, 2020.
(60) Provisional application No. 63/071,029, filed on Aug. 27, 2020.

(51) Int. Cl. A61K 33/30; A61K 31/355; A23L 33/15; A23L 33/00; A23L 29/00

(52) U.S. Cl. CPC A61K 33/30; A61K 31/355; A23L 29/035; A23L 33/30; A23L 33/15

(57) ABSTRACT

The present invention provides compositions and methods for the treatment and prevention of COVID-19. The disclosed compositions comprise a combination of the natural compounds zinc, quercetin, vitamin E, and epigallocatechin gallate (EGCG), and are formulated for use in treating COVID-19, reducing the severity of disease, and enhancing the immune response to vaccination for COVID-19.

### CROSS-REFERENCE TO RELATED APPLICATIONS

This application claims priority to U.S. Provisional Application No. 63/029,157 filed on May 22, 2020 and to U.S. Provisional Application No. 63/071,029 filed on Aug. 27, 2020, the contents of which are incorporated by reference in their entireties.

### INTRODUCTION

The U.S. and global COVID-19 pandemic have raised the critical need for rapidly performed, pragmatic randomized trials to test new compounds to reduce risk of transmission, improve clinical outcomes, and augment responsiveness to vaccines. COVID-19 (i.e., coronavirus disease 2019) is an infectious disease caused by severe acute respiratory syndrome coronavirus 2 (SARS-CoV-2). Common symptoms include fever, cough, fatigue, shortness of breath, and loss of smell and taste. While the majority of cases result in mild symptoms, some progress to significant respiratory symptoms, effects in other tissues, need for hospitalization, cytokine storm and acute respiratory distress syndrome (ARDS), acute kidney injury and other multi-organ effects, and death. The precipitous scope and scale of hospitalizations, ICU admissions, and deaths have appropriately altered the traditional multi-year approach to test promising agents for clinical efficacy. While pharmacologic agents such as chloroquine, hydroxychloroquine, and remdesivir are each being tested, there is at least similar if not greater compelling evidence for potential benefits of several natural immune-modulating compounds and with far more attractive safety profiles.

### SUMMARY

In one aspect, the present invention provides compositions comprising at least two components selected from the group consisting of zinc, quercetin, vitamin E, and epigallocatechin gallate (EGCG). The composition may be comprised within a food or nutritional supplement.

In a second aspect, dose packs including a multiplicity of doses of at least two components selected from zinc, quercetin, vitamin E, and epigallocatechin gallate (EGCG) are provided. The dose pack is configured to isolate doses of the at least two components.

In another aspect, the present invention provides methods of administering the compositions and dose packs described herein to a subject.

In a further aspect, the present invention provides methods of administering to a subject at least three components selected from the group consisting of zinc, quercetin, vitamin E, and EGCG.

In another aspect, the present invention provides methods of treating a subject for COVID-19. The methods comprise (a) selecting a subject who has tested positive for COVID-19; and (b) administering to the subject at least three components selected from the group consisting of zinc, quercetin, vitamin E, and EGCG.

In another aspect, the present invention provides methods of increasing the immune response to a vaccine for COVID-19. The methods comprise (a) selecting a subject who will be receiving a vaccine for COVID-19; (b) administering to the subject at least two components selected from the group consisting of zinc, quercetin, vitamin E, and EGCG; and (c) administering the vaccine for COVID-19.

### DETAILED DESCRIPTION

A combination of natural compounds for the treatment of COVID-19, including at least two, three or all four of zinc, quercetin vitamin E, and EGCG, is provided herein. The inventors expect this combination of compounds to lower risk of acquiring COVID-19, reduce the clinical progression of COVID-19, to reduce symptoms associated with or illness caused by COVID-19, and increase the immune response to a vaccine for COVID-19. The inventors will test the ability of this combination of compounds to produce these effects, as well as additional benefits, in clinical trials. Further, they will experimentally assess whether the individual compounds have additive or synergistic effects when used in combination. COVID-19 is the disease state caused by SARS-CoV-2 and these two terms will be used interchangeably.

### Compositions:

The present invention provides compositions comprising at least two components selected from the group consisting of zinc, quercetin, vitamin E, and epigallocatechin gallate (EGCG). In some embodiments, the compositions comprise at least three of zinc, quercetin, vitamin E, and EGCG. In some embodiments, the compositions comprise all four of the components (i.e., zinc, quercetin, vitamin E, and EGCG).

Zinc is a metal element and an essential micronutrient that is crucial to almost every aspect of human health. Zinc may be provided as zinc oxide, a zinc salt (e.g., zinc gluconate, zinc picolinate), or another form of zinc that is easily absorbed by the body. Quercetin is a plant flavonol from the flavonoid group of polyphenols, and is found in many fruits, vegetables, leaves, seeds, and grains. Vitamin E is a group of eight fat soluble compounds that include four tocopherols and four tocotrienols. Various forms of Vitamin E may be used with the present invention and may comprise various relative amounts of particular tocols, such as alpha-tocopherolacetate. EGCG is the ester of epigallocatechin and gallic acid, and is the most abundant type of catechin found in tea.

In some embodiments, the composition is provided as a food product. Here, the term “food” is used in a broad sense and encompasses food and drink for humans as well as food and drink for animals (i.e., a feed). Preferably, the food product is suitable for human consumption. The food product may be in the form of a liquid, solid, or suspension. When in the form of a food product, the composition may further comprise or be used in conjunction with one or more of: a nutritionally acceptable carrier, a nutritionally acceptable diluent, a nutritionally acceptable excipient, a nutritionally acceptable adjuvant, and a nutritionally active ingredient. Exemplary food products include a beverage powder, a soy milk, a coffee beverage, a fruit juice, a beverage comprising whey protein, an herbal tea, a cocoa drink, a milk drink, a lactic acid bacteria drink, a yogurt, a cheese, an ice cream, a dessert, a confectionery, a biscuit, a cake, a cake mix, a fruit filling, an icing, a snack food, or a nutritional bar or supplement.

In some embodiments, the composition comprises 10-75 mg of zinc, 100-1500 mg of quercetin, 50-800 mg of vitamin E, and/or 300-600 mg of EGCG. In one suitable embodiment, the composition may contain 60 mg Zinc, 1000 mg quercetin, 200 mg Vitamin E, and 500 mg EGCG.

The components used in the compositions of the present may be in any form that is suitable for administration to a human subject. Importantly, claimed components are all Generally Regarded as Safe (GRAS) by the U.S. Food and Drug Administration, making them excellent candidates for use in the combination therapies described herein.

In some embodiments, the compositions are formulated into a dosage unit for daily administration. As used herein, the term “dosage unit” refers to an individual portion for therapeutic use. Suitable dosage units include, for example, a tablet, pill, ampule, capsule, cachet, or a single dose sachet of a powder or liquid.

### Dose Packs:

Also provided herein are dose packs including a multiplicity of doses of at least two components selected from the group consisting of zinc, quercetin, vitamin E, and epigallocatechin gallate (EGCG). “Dose pack,” as used herein, means a product for the containment of one or more dosage form, such that each dose that should be taken by a subject at a single point in time is packaged in a single compartment. A “dose form” or “dosage form” refers to a physical composition such as a tablet, pill, ampule, capsule, cachet, or a single dose sachet of a powder or liquid comprising the indicated components that form an individual portion for therapeutic use. A “dose”, as used herein, means a quantity of a therapeutic taken or recommended to be taken at a particular time. A dose may include more than one dosage form. For example, in some embodiments, the dosage form comprises 10-75 mg of zinc, 100-1500 mg of quercetin, 50-800 mg of vitamin E, and/or 300-600 mg of EGCG.

The dose packs are configured to isolate individual, suitably daily, doses of the at least two components. In some embodiments, each dose is for daily administration. The dose packs may include doses that contain two, three, or all four of the indicated components. In some embodiments the dose packs contain two, three, or all four of the indicated components along with one or more carrier or excipient. In some embodiments, additional components may be included in the doses of the dose packs. Suitable additional components for inclusion in the dose packs include other natural components such as Vitamin C, Vitamin D, or additional antioxidants as well as anti-viral pharmaceuticals, immune suppressants or immune stimulators. In some embodiments, the at least three of the components are included in the multiplicity of doses. In some embodiments, each of the multiplicity of doses contains all four components.

Each dose within the dose pack may have the at least two components comprised in a unitary dosage form or may have at least two dosage forms per dose to contain the at least two components. In other words, in some cases, each dose may contain a unitary formulation containing the at least two components. Alternatively, each dose may be comprised of at least two dosage forms, wherein each dosage form contains at least one component. In some embodiments, the at least two components are comprised in a unitary dosage form in each dose. In some embodiments, the at least two components are comprised in at least two dosage forms in each dose.

The dose pack may include any suitable container for the components that make up a single dose. In some embodiments, the dose pack comprises a blister pack configured to isolate each dose. The blister pack may be configured to isolate a unitary formulation comprising the at least three components. Alternatively, the blister pack may be configured to isolate a higher number of formulations, such as a binary formulation, either in a single chamber or separate chambers of the blister pack to make up a single dose in the dose pack.

### Methods:

The present invention provides methods of administering the compositions described herein to a subject. As used herein, the terms “administering” and “administration” refer to any method of providing a pharmaceutical preparation to a subject. Such methods are well known to those skilled in the art and include, but are not limited to, oral administration, gastro-intestinal administration, and nasogastric administration (e.g. via a nasogastric or enteral feeding tube). Administration can be continuous or intermittent. For example, continuous administration may be accomplished using a feeding tube. Intermittent administration may be oral administration, e.g., via once or twice daily tablets.

As used herein, “subject” or “patient” refers to mammals and non-mammals. A “mammal” may be any member of the class Mammalia including, but not limited to, humans, non-human primates (e.g., chimpanzees, other apes, and monkey species), farm animals (e.g., cattle, horses, sheep, goats, and swine), domestic animals (e.g., rabbits, dogs, and cats), or laboratory animals including rodents (e.g., rats, mice, and guinea pigs). Examples of non-mammals include, but are not limited to, birds, and the like. The term “subject” does not denote a particular age or sex. In some embodiments, a subject is a mammal, preferably a human. In some embodiments, the subject has tested positive for COVID-19. In some embodiments, the subject has been exposed to COVID-19.

The present invention also provides methods of administering to a subject at least three components selected from the group consisting of zinc, quercetin, vitamin E, and EGCG. In some embodiments, the methods inhibit viral infection or replication in the subject. In other embodiments, at least two of the components are administered to increase the immune response to a vaccine, the vaccine may be directed to protect against SARS-CoV-2 infection.

In some embodiments, the viral infection is COVID-19. In some embodiments, the subject has tested positive for COVID-19. In some embodiments, the subject has been exposed to COVID-19 or is at high risk for acquiring (becoming infected with) COVID-19. High-risk populations include people who are more likely to be exposed to COVID-19 including, without limitation, people housed in nursing homes, jails, and urban areas, as well as people who work on the front lines, e.g., healthcare providers. In some embodiments, the subject will be receiving a vaccine for COVID-19 and may be at risk for having a lower than optimal immune response to such a vaccine. Populations at such risk may include, without limitation, older adults or people with obesity, diabetes or high blood pressure, for example.

The present invention also provides methods of treating a subject for COVID-19. The methods comprise (a) selecting a subject who has tested positive for COVID-19; and (b) administering to the subject at least three components selected from the group consisting of zinc, quercetin, vitamin E, and EGCG. As used herein, “treating” or “treatment” describes the management and care of a subject for the purpose of combating COVID-19. Treating includes the administration of zinc, quercetin, vitamin E, and/or EGCG to prevent the onset of the symptoms or complications, to alleviate the symptoms or complications, or to eliminate the viral infection altogether.

The present invention also provides methods for reducing the risk of a subject of acquiring COVID-19. The methods comprise (a) selecting a subject who may have been exposed or otherwise be at risk for acquiring COVID-19; and (b) administering to the subject at least three components selected from the group consisting of zinc, quercetin, vitamin E, and EGCG. As used herein, reducing the risk of acquiring COVID-19 means decreasing the probability of becoming infected even if exposed to high risk situations or decreasing the risk of having adverse consequences of infection or decreasing the risk of requiring hospitalization. The administration of zinc, quercetin, vitamin E, and/or EGCG may be used to prevent, limit or reduce the severity of COVID-19 infection.

The present invention also provides methods of increasing the immune response to a vaccine for COVID-19. The methods comprise (a) selecting a subject who will be receiving a vaccine for COVID-19; (b) administering to the subject at least two components selected from the group consisting of zinc, quercetin, vitamin E, and EGCG; and (c) administering the vaccine for COVID-19. As used herein, “increasing the immune response” describes increasing the antibody titer, the T cell memory response, the number of circulating T and B cells or the rapidity of generation of such an immune response to a COVID-19 vaccine. A “vaccine for COVID-19” may refer to any composition that is capable of inducing an immune response against SARS-CoV-2 in a subject. The vaccine used with the present invention may be of any suitable composition directed against SARS-CoV-2 including, without limitation, a DNA vaccine, an inactivated viral vaccine, or an attenuated live vaccine.

The supplementation with at least two of the indicated components may be administered prior to vaccine administration, i.e., to “prime” the immune system. For example, the supplements may be administered 2-4 weeks prior to vaccine administration. Optionally, supplementation may be continued until the antibody titer reaches a maximum level, which takes about 3 weeks with typical vaccines. Generally the administration of the at least two components is daily administration, but may also be provided in other dosage forms such as two per day or other day.

In some embodiments, the subject may be at risk for an attenuated immune response to the COVID-19 vaccine. As used herein, the term “attenuated immune response” refers to an immune response that is weaker than the average immune response. Many factors contribute to how well a subject responds to a given vaccine including, for example, genetics, immune status, age, health, and nutritional status. Immunocompromised subjects may also be included as individual with attenuated immune responses and include those subjects who are immunocompromised due to a disease such as HIV or those on immunosuppressive therapies to combat an unrelated auto-immune disease or after transplantation.

In some embodiments, the at least two components selected from the group consisting of zinc, quercetin, vitamin E, and EGCG are administered at least daily for at least one week prior to the vaccine administration. In some embodiments, the at least two components are administered at least daily for at least two weeks prior to the vaccine administration. In some embodiments, at least three of the components selected from the group consisting of zinc, quercetin, vitamin E, and EGCG are administered. In still other embodiments all four of the components are admininstered.

All of the combination treatments of the present invention are designed to curtail viral infection, particularly infection by SARS-CoV-2. Thus, in some embodiments, the methods of the present invention reduce hospitalization time, intubation time, and/or disease duration of the subject. In some embodiments, the methods curtail the clinical progression and/or reduces the symptom severity of COVID-19. In some embodiments, the methods curtail the risk of transmitting an infection or curtail the risk of being infected by COVID-19. In some embodiments, the methods reduce symptom severity and/or curtail clinical progression if a previously uninfected subject does become infected by COVID-19. In some embodiments, the methods increase the immune response if a subject receives a vaccine for COVID-19. As used herein, the term “curtails clinical progression” refers to the ability of a treatment to prevent hospitalization or reduce the length of hospitalization, need for supplemental oxygen, need for intubation or the length of time intubated, and/or death of a subject. As used herein, the term “reduces symptom severity” refers to the ability of a treatment to reduce or eliminate any symptom of COVID-19 as compared to treatment with a placebo. Exemplary symptoms of COVID-19 include, without limitation, cough, shortness of breath or difficulty breathing, pain or pressure in the chest, fever, chills, muscle pain, sore throat, loss of taste or smell, confusion, inability to wake or stay awake, bluish lips or face, rashes or other dermatologic effects such as COVID toe, gastro-intestinal effects such as nausea, diarrhea and vomiting and endothelial dysfunction which may include neovascularization or angiogenesis. As used herein, the term “curtails the risk of transmitting an infection” refers to the ability of a treatment to reduce the likelihood that the subject having COVID-19 passes the infection to a previously uninfected individual. As used herein, the term “curtails the risk of being infected by COVID-19” refers to the ability of a treatment to reduce the likelihood that an infection is established in an uninfected subject upon exposure to SARS-CoV-2 or to increase the efficacy or protection level provided by vaccination against SARS-CoV-2. In some embodiments of the present methods, all four compounds (i.e., zinc, quercetin, vitamin E, and EGCG) are administered to the subject.

In some embodiments, the compounds are administered to the subject at a particular dosage. For example, in some embodiments, zinc is administered to the subject at 10-75 mg/d, preferably at about 60 mg/d. In some embodiments, quercetin is administered to the subject at 100-1500 mg/d, or at 100-1000 mg/d twice daily (bid), preferably at about 500 mg bid. In some embodiments, vitamin E is administered to the subject at 50-800 mg/d, preferably at about 200 mg/d. In some embodiments, EGCG is administered to the subject at 300-1600 mg/d, preferably at about 500 mg/d.

In some embodiments, subject is considered to be at high risk of severe illness from COVID-19. Subjects that are considered to be at high risk include those who are over the age of 60, as well as those who are over the age of 45 and have a serious underlying medical condition, such as diabetes, hypertension, cardiovascular disease, heart failure, obesity (BMI>30 kg/m2), chronic kidney disease, and/or cancer, or a combination of a subset of these conditions.

The four compounds used in the compositions and methods of the present invention were selected based on five primary lines of mechanistic evidence. First, these compounds are thought to have specific activity against COVID-19 proteins, including interfering with binding of the virus S-protein to the host ACE2 receptor (i.e., the port of entry to the host cell), inhibiting the RNA-dependent RNA polymerase of the virus (which is necessary for viral replication), and inhibiting the 3C-like protease (3CLpro; a key enzyme in the viral replication pathway) activity of the virus. See the section titled “Evidence for specific activity against COVID-19 proteins” for details. Thus, in some embodiments, the methods inhibit viral entry into cells or viral reproduction in the subject.

Second, the compounds disclosed herein have been shown to enhance pathogen killing, e.g., by improving normal immune responses and by increasing T cell function. See the section titled “Evidence for general augmented pathogen killing” for details. Thus, in some embodiments, the methods increase T cell function in the subject.

Third, the compounds disclosed herein have been shown to reduce activation of the primary pathways that contribute to poor outcomes and lung damage in COVID-19 patients. See the section titled “Evidence for reduced cytokine storm and lung injury” for details. Thus, in some embodiments, the methods reduce inflammation, cytokine levels, and/or reactive oxygen species in the subject. In some embodiments, the methods reduce tissue damage in the subject.

Fourth, the compounds disclosed herein have been shown to normalize and/or improve vascular endothelial function. The vascular endothelium is a monolayer of cells at the interface between blood and tissue. These cells are particularly and unusually susceptible to infection and damage from COVID-19, as compared to other respiratory viruses,. Thus, in some embodiments, the administration of the composition improves endothelial function and/or reduces harms from COVID-19 in non-respiratory tissues including the blood vessels, heart, kidney, brain, and/or extremities (e.g., toes), among other tissues.

Fifth, the compounds have complementary and synergistic mechanistic effects that, together, improve the ability of the compounds to curtail COVID-19 infection. While quercetin and/or EGCG reduce activation of the primary pathways that contribute to poor outcomes and lung damage in COVID-19 patients, the anti-inflammatory effects of these compounds may also contribute to reduced T cell function and thereby diminish the beneficial immune responses against the virus. Zinc and/or vitamin E augment T cell function, allowing for preserved T cell function and/or pathogen response in subjects that are also being treated with quercetin and/or EGCG, thereby both fighting the virus and reducing activation of the inflammatory pathways that contribute to poor outcomes and lung damage. This increased T cell function may result in enhanced T cell dependent CTL response and an increased Th1:Th2 ratio.

Sixth, the compounds have been shown to increase the immune response to vaccines. Specifically, vitamin E has been shown to increase antibody titer to hepatitis B and tetanus vaccines in humans (JAMA (1997) 277(17):1380), and zinc has been shown to increase antibody response to the tetanus vaccine (Am J Med (1981) 70(5):1001-4).

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 0.01% to 5%, it is intended that values such as 0.025% to 0.50%, 0.10% to 1.0%, or 0.025% to 0.075%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference in their entirety, unless explicitly indicated otherwise. The present disclosure shall control in the event there are any disparities between any definitions and/or description found in the cited references.

Unless otherwise specified or indicated by context, the terms “a”, “an”, and “the” mean “one or more.” For example, “a protein” or “an RNA” should be interpreted to mean “one or more proteins” or “one or more RNAs,” respectively.

The following examples are meant only to be illustrative and are not meant as limitations on the scope of the invention or of the appended claims.

### EXAMPLES

In the following Example, the inventors (1) detail the rationale for the selection of the compounds zinc, quercetin, vitamin E, and EGCG for the treatment of COVID-19 and (2) outline two prophetic clinical trials designed to test the use of these compounds to treat and/or prevent COVID-19.

### Rationale for the Selection of Zinc, Quercetin Vitamin E, and EGCG

Preclinical studies and human trials support benefits of specific natural compounds for immune responses and outcomes of acute respiratory infections from SARS-CoV, other coronaviruses, influenza, and other viral pathogens. While several candidates have immune-modulating effects (e.g., vitamin C, D, sulforaphane, selenium), the four compounds with greatest promise for COVID-19 are zinc, quercetin, vitamin E, and EGCG, based on the mechanisms of action of these compounds and the clinical responses observed in viral infections (Table 1). There is compelling evidence that these compounds, in combination, would provide benefits across five complementary pathways that play a critical role in COVID-19:
1. Evidence for specific activity against COVID-19 proteins, including interfering with binding of the virus S-protein to the host ACE2 receptor (the port of entry to the host cell), inhibiting the RNA-dependent RNA polymerase of the virus (needed for viral replication), and inhibiting the 3C-like protease (3CL^{pro}) activity of the virus (a key enzyme in the viral replication pathway).
2. Evidence for general augmented pathogen killing, including improved normal immune responses and increased T cell function.
3. Evidence for reductions in excessive inflammation, cytokine storm, reactive oxygen species, and corresponding lung damage, which are crucial primary pathways for poor outcomes with COVID-19 including the need for hospitalization, mechanical ventilation, and risk of death.
4. Evidence for normalized and/or improved vascular endothelial function, a tissue type that is particularly and unusually susceptible to infection and damage from COVID-19. Endothelial infection and damage from COVID-19 appears central to damage to non-respiratory tissues including the blood vessels, heart, kidney, brain, extremities (e.g., toes), and other tissues.
5. Evidence for improved clinical outcomes in viral respiratory infections in human clinical trials.
6. Evidence for augmenting the immune response to vaccines given to prevent viral infections.
   1. Evidence for Specific Activity against COVID-19 Proteins

In preclinical studies, zinc inhibits the RNA-dependent RNA polymerase of SARS-CoV, which is highly homologous with the RNA-dependent RNA polymerase of COVID-19¹ and is necessary for viral replication.² In computer modeling of diverse drugs and natural compounds, quercetin was identified as a top potential antagonist to the COVID-19 S-protein, which binds to the host cell ACE2 receptor as the entry way for infection;³ this finding is supported by preclinical studies in which quercetin binds the SARS-CoV ACE2 receptor.⁴ SARS-CoV-2 and SARS-CoV also share an almost identical main protease (3CL^{pro}), a key enzyme for viral replication.⁵ Quercetin inhibits the (3CLP″) of both SARS-CoV and MERS, and also the (Pl^{pro}) protease of SARS-CoV;⁵ and inhibits viral replication and risk of acute infection in animal models of influenza.^{6,7} EGCG also inhibits the (3CL^{pro}) activity of SARS-CoV and inhibits infectivity of a wide range of other viruses including influenza, adenovirus, Ebola, and others through high affinity, nonspecific binding to viral surface proteins.⁸⁻¹⁰

### 2. Evidence for General Augmented Pathogen Killing

Zinc and vitamin E are known to be required for optimal immune function, with low intakes leading to less effective immune responses and higher susceptibility to respiratory infection.^{11,12} Low serum zinc levels are associated with higher risk of coronavirus 229E and OC34 infections;^{13,14} while zinc supplementation increases T cell function in older adults and reduces lower respiratory infection.¹⁵ Vitamin E reduces viral load in viral lung infections,¹⁶ and increases resistance to and reduces pathogenesis of influenza.^{16,17} Vitamin E supplementation also increases T cell function in older adults;¹⁸ while SARS-CoV infection reduces vitamin E levels in the lung.^{19,20}

### 3. Evidence for Reduced Cytokine Storm and Lung Injury

Beyond effects to inhibit viral entry, interfere with viral replication, and augment T cell function and pathogen killing, these compounds have complementary effects to reduce excessive inflammation and tissue damage from excess cytokines and reactive oxygen species (ROS)²¹ that are central to the acute lung injury and failure, ICU admissions, need for mechanical ventilation, and deaths from COVID-19.²² Quercetin, for example, inhibits cytokine storm and reduces lung injury induced by rhinovirus infection in mice^{23,24} and reduces superoxide radical formation, lung pathology, and overall risk of infection induced by influenza.^{6,7,25} Vitamin E reduces viral load, dampens excessive lung inflammatory responses to viral infection, and increases survival in viral pneumonia.¹⁶⁻¹⁸ EGCG down-regulates pro-inflammatory cytokines in acute viral respiratory infection;²⁶ and suppresses TLR4 and NF-κB protein levels, ROS, and acute tissue injury in multiple diverse models of lung injury.²⁷⁻³³ Evidence from in vitro culture with SARS-CoV reproduction enzymes^{2,34} computer modelling,³ and systematic reviews of candidate compounds further supports benefits of these agents for COVID-19.³⁵

4. Evidence for Normalized and/or Improved Vascular Endothelial Function

The vascular endothelium is a monolayer of cells at the interface between blood and tissue. These cells are particularly and unusually susceptible to infection and damage from COVID-19, as compared to other respiratory viruses. Thus, in some embodiments, the administration of the composition improves endothelial function and/or reduces harms from COVID-19 in non-respiratory tissues including the blood vessels, heart, kidney, brain, and/or extremities (e.g., toes), among other tissues.

Several of the four natural compounds may have complementary effects on restoring and improving vascular endothelial function. In a meta-analyses of randomized controlled trials, vitamin E was shown to improve vascular endothelial function (Obes Rev. February 2014; 15(2):107-116; Br J Nutr. Apr. 28, 2015; 113(8):1182-1194). EGCG induces protective nitric oxide (NO) release and reduces endothelin-1 production in endothelial cells (J Cardiovasc Pharmacol. April 2020; 75(4):292-298). EGCG also lowers levels of biomarkers of endothelial dysfunction such as ICAM-1 and VCAM-1; increases NO bioavailability through lowering of asymmetric dimethylarginine, the endogenous NO inhibitor; and reduces excessive endothelial oxidative stress through effects on the Nrf2/HO-1 pathway (J Cardiovasc Pharmacol. April 2020; 75(4):292-298). Quercetin also exerts several beneficial effects in vitro on endothelial cell pathways (Free Radic Res. October 2006; 40(10):1054-1065), with some although still mixed evidence for benefits on endothelial dysfunction in randomized controlled trials (Curr Pharm Des. 2019; 25(12):1372-1384). Restoring endothelial function and reducing endothelial inflammation appears especially germane for COVID-19, which exhibits a highly unusual predilection to infect and harm, in addition to respiratory epithelial cells, vascular endothelial cells and cause inflammation, pathogenic angiogenesis, and eventually cell death (N Engl J Med. May 21, 2020). These unusual effects may explain many of the extra-respiratory manifestations that have been seen with COVID-19, including risk of kidney failure, stroke, inflammatory vasculitis in children, and lower extremity abnormalities (e.g., ‘COVID toe’).

### 5. Evidence for Improved Clinical Outcomes in Human Trials

Randomized placebo-controlled trials (RCTs) support benefits of these compounds for acute respiratory infections in humans.³⁶⁻⁴¹ Pooling 17 RCTs (N=2,121), short-term zinc supplementation at the onset of symptoms of the common cold (often caused by coronaviruses) reduced days of illness (−1.65 days, −2.50 to −0.81).³⁶ A significant association between serum zinc levels and risk for pneumonia including viral pneumonias was observed in elderly nursing home residents.¹³ Zinc supplementation in a double-blind, placebo controlled trial reduced acute respiratory infections in the elderly.³⁷ In a placebo-controlled trial of vitamin E (N=642), treatment reduced the risk of total acute respiratory infections and, in secondary analysis, of colds (often caused by strains of coronavirus).³⁸ In a community-based trial of adults (N=1,002, age 18-85 years), quercetin supplementation reduced severity and duration of upper respiratory infection in the subset of subjects 40+ years of age (N=325) by 36% and 31% respectively.³⁹ In 2 small RCTs (N=108, 197), EGCG supplementation reduced symptoms from colds or influenza by 32% (P=0.035) and days with symptoms by 36% (p<0.002); and reduced clinically diagnosed influenza infection by 75%) (OR=0.25, 95% CI=0.07-0.76) and days free from influenza infection by 73% (HR=0.27, 95% CI=0.09-0.84).⁴⁰

### 6. Evidence for Augmenting the Immune Response

Zinc and vitamin E are known to be required for optimal immune function, with low intakes leading to less effective immune responses and higher susceptibility to respiratory infection.^{11,12} Low serum zinc levels are associated with higher risk of coronavirus 229E and OC34 infections;^{13,14} while zinc supplementation increases T cell function in older adults and reduces lower respiratory infection.¹⁵ Vitamin E reduces viral load in viral lung infections,¹⁶ and increases resistance to and reduces pathogenesis of influenza.^{16,17} Vitamin E supplementation also increases T cell function in older adults¹⁸ while SARS-CoV infection reduces vitamin E levels in the lung.^{19,20} EGCG supplementation reduced symptoms from colds or influenza by 32% and days with symptoms by 36%; and reduced clinically diagnosed influenza infection by 75% and days free from influenza infection by 73%.⁴⁰ In an observational study among 2,050 Japanese children, higher intake of green tea was associated with 46% lower risk of influenza⁴⁰ In a small randomized control trial (N=40), quercetin supplementation significantly reduced the incidence of acute respiratory infections (P=0.004).⁴¹ In a large randomized control trial (N=1,002), quercetin supplementation had no overall significant effect on rates of upper respiratory infections, but reduced symptom severity (−36%) and total sick days (−31%) in a secondary analysis among older subjects (age 40+) with higher self-reported fitness.³⁹ Both older adults and individuals with obesity have been shown to have reduced immune responsiveness to a range of vaccines, limiting the effectiveness of the vaccine for both the individual and for overall herd immunity. Several of the selected compounds have been shown to help improve responsiveness to vaccines. In older adults, vitamin E supplementation improves the in vivo delayed-type hypersensitivity skin response as well as antibody production in response to vaccination, mediated through increased production of interleukin(IL)-2, leading to enhanced proliferation of T cells, and through reduced production of prostaglandin E(2), a T-cell suppressive factor, as a result of a decreased peroxynitrite formation (JAMA. May 7, 1997; 277(17):1380-1386; Immunol Rev. June 2005; 205:269-284). Vitamin E increased both cell-dividing and IL-producing capacities of naive T cells, but not memory T cells. The vitamin E-induced enhancement of immune functions in the aged was associated with significant improvement in resistance to influenza infection in aged mice and a reduced risk of acquiring upper respiratory infections in nursing home residents (JAMA. May 7, 1997; 277(17):1380-1386). Mild and otherwise subclinical zinc deficiency, which increases substantially with age, is associated with abnormalities in adaptive immunity, in T cell activation, and in T cell polarization.^{44,45} This is associated in older adults with a non-specific pre-activation of T cells and a decreased responsiveness to vaccination, termed “immunosenescence.” Zinc supplementation restores many of these functional abnormalities, including antibody responses to vaccines (Biogerontology. October-December 2006; 7(5-6):421-428; J Trace Elem Med Biol. January 2015; 29:24-30; Am J Med. May 1981; 70(5):1001-1004). In experimental models, EGCG has also potentiated the immune response to vaccination, including with influenza vaccine (Cancer Lett. Jul. 1, 2018; 425:152-163; J Nat Med. January 2017; 71(1):68-75; Cancer Res. Jan. 15, 2007; 67(2):802-811); while quercetin improves the response to respiratory syndrome virus-1 vaccine in a large mammal model (Vaccine. Apr. 23, 2020; 38(19):3570-3581). In addition, these compounds may be synergistic: for example, the combination of vitamin E and EGCG may synergistically enhance adaptive B-cell and CD4(+) and CD8(+) T-cell responses (Immunology. August 2016; 148(4):352-362). Together, these findings support the complementary roles of vitamin E and zinc, and possibly also EGCG and quercetin, for normalizing immune responsiveness to vaccines in at-risk individuals.

### Conclusion

Together, the combined, complementary effects of these natural compounds is expected to boost the immune system, increase vaccine efficacy, and reduce excessive inflammatory responses in the lung. Thus, these compounds help bend the curve of severe illness from COVID-19, providing much needed partial relief to hospital systems, shuttered economies, and global populations. The combined, complementary effects of these natural compounds prevents transmission, curtails viral infection in previously uninfected individuals, and boosts the immune response to a vaccine for COVID-19. Given the urgency of the ongoing and growing national and global pandemic, it is of critical importance to determine the clinical efficacy of these compounds for COVID-19. Such natural compounds may also be particularly beneficial in older adults and/or individuals with obesity, diabetes, hypertenion, and other chronic conditions, who are more likely to exhibit micronutrient deficiency, impaired cell-mediated immune response, and chronic low-grade systematic inflammation; and who have highest rates of poor outcomes from COVID-19. Their shared and complementary effects render a combination therapy most promising, as each compound by itself may have insufficient efficacy to be detected in a reasonably sized clinical trial. Importantly, the safety profile of these nutrients all are Generally Regarded as Safe (GRAS) by the U.S. Food and Drug Administration makes combination therapy highly attractive.

TABLE 1
Evidence pertinent to COVID-19 for immune-modulating effects of the natural compounds to be tested in this trial.*

| | Evidence for specific | Evidence for general augmented | Evidence for reduced | |
|---|---|---|---|---|
| | activity against COVID-19 | immune function and resistance to | cytokine storm and | Evidence for improved clinical |
| Compound | proteins | viral respiratory infections | lung injury | outcomes in human trials |
| Zinc | Inhibits the RNA-dependent | Increases T cell function in older | Reduces inflammation. | In a meta-analysis of 17 RCTs |
| | RNA polymerase needed for | adults and reduces lower | | (N = 2,121), short-term |
| | viral replication (studies | respiratory infection.¹⁵ | | zinc supplementation at the |
| | with SAR-CoV).² Interferes | 20-30% of older adults have low | | onset of symptoms of the common |
| | with proteolytic process. | serum zinc, which is associated | | cold (often caused by |
| | | with higher incidence of pneumonia | | coronaviruses) reduced days of |
| | | including from coronavirus 229E | | illness by 1.65 days (95% CI: |
| | | and OC34.^{13, 14} | | 0.81 to 2.50).³⁶ |
| | | Needed for normal function of | | |
| | | immune cells. | | |
| Quercetin | Antagonist to the binding of | Inhibits viral replication and | In animal models with | In a small RCT (N = 40), |
| | the SARS-CoV S-protein to | risk of acute infection in animal | increased inflammatory | quercetin supplementation |
| | ACE2 receptor on the host | models of influenza.^{6, 7} | cytokines (such as | significantly reduced the |
| | cells, the entry way for | | rhinovirus infection | incidence of acute respiratory |
| | COVID-19 (studies with | | in COPD), reduces | infections (P = 0.004).⁴¹ |
| | SARS-CoV).⁴ | | inflammation and lung | In a large RCT (N = 1,002), |
| | Identified in computer | | damage.²³⁻²⁵ | quercetin supplementation had |
| | modeling of diverse drugs and | | Reduces superoxide | no overall significant effect |
| | other compounds as a top | | radicals, infiltrating | on rates of upper respiratory |
| | candidate to interfere with | | cells, and lung injury | infections, but reduced symptom |
| | binding of the virus S-protein | | in animal models of | severity (−36%) and total sick days |
| | to ACE2 receptor on the host | | influenza.^{6, 25} | (−31%) in a secondary analysis |
| | cells, the entry way for | | | among older subjects (age 40+) |
| | COVID-19.³ | | | with higher self-reported |
| | Inhibits the 3C-like protease | | | fitness.³⁹ |
| | (3CL^{pro}) activity of | | | |
| | COVID-19.⁵ | | | |
| Vitamin E | Viral infection including with | Increases T cell function in | Reduces inflammatory | Decreased colds in older adults |
| | SARS-CoV results in lower | older adults.¹⁸ | cytokine production in | (secondary outcome).³⁸ |
| | level of vitamin E in | Reduces viral load in viral | response to influenza.¹⁷ | Decreased pneumonia in a subset |
| | lung.^{19, 20} | lung infection.¹⁶ | Increases survival from | of nursing home residents with |
| | | Increases resistance to and | strep pneumonia infection | polymorphism.⁴³ |
| | | reduces pathogenesis of influenza | in old mice by reducing | |
| | | infection in older mice.^{16, 17} | neutrophil migration and | |
| | | | inflammatory cytokines.⁴² | |
| | | Necessary for normal immune | | |
| | | response.¹² | | |
| EGCG | Inhibits the (3CL^{pro}) activity | Inhibits infectivity of a wide | Down-regulatcs pro- | In 2 small RCTs (N = 108, |
| | of SARS-CoV, a key enzyme | range of viruses through high | inflammatoiy cytokines, such | 197), EGCG supplementation |
| | in the viral replication | affinity but nonspecific binding | as TNF-α, IL-6 and IL-8. | reduced symptoms from colds or |
| | pathway. | to viral surface proteins, including | in acute viral respiratory | influenza by 32% (P = 0.035) |
| | | for influenza, adenovirus. Ebola, and | infection.²⁶ | and days with symptoms by |
| | | others.⁸⁻¹⁰ | Suppresses inflammatory | 36% (p < 0.002); and reduced |
| | | | mediators. TLR4 and NF-κB | clinically diagnosed influenza |
| | | | protein levels, ROS | infection by 75%) (OR = 0.25, |
| | | | production, acute lung | 95% CI = 0.07-0.76) and days |
| | | | injury, and apoptosis in | free from influenza infection by |
| | | | other models of lung | 73% (HR = 0.27, 95% CI = |
| | | | injury.²⁷⁻³³ | 0.09-0.84).⁴⁰ |
| | | | | In an observational study among |
| | | | | 2,050 Japanese children, higher |
| | | | | intake of green tea was associated |
| | | | | with 46% lower risk of influenza |
| | | | | (OR = 0.54, 95% CI = |
| | | | | 0.30-0.94).⁴⁰ |

*Additional preclinical evidence suggests some potential for benefits of other selected natural compounds, including for example vitamin C and D. Upon our detailed review, the evidence supporting these other agents for treatment of acute viral respiratory infections was not considered robust enough or suggestive for benefits for COVID-19.
RCT = randomized controlled trial

### Example 1

Specific Aim: A first trial will test a combination of 4 natural compounds for efficacy in reducing the clinical progression of COVID-19, defined as first of hospitalization or death for outpatients, and first of mechanical ventilation or death for inpatients. A second trial will test the efficacy of the disclosed combinations of compounds for reduction of primary infection in very high-risk populations, such as those in nursing homes, jails, urban areas, or other locations, as well as in healthcare providers on the front lines. Specifically, these two studies will test the potential benefits of the compounds for prevention of infection and for reducing the severity of illness. The major difference in the two proposed experiments is the subject population being given the treatment.

Hypothesis: Oral zinc (60 mg/d)+quercetin (500 mg bid)+Vitamin E (200 mg/d)+EGCG (500 mg/d), in combination, will reduce risk of clinical progression of COVID-19 and reduce transmission of the virus.

Population: For experiment 1, the subjects will all be outpatients, nursing home/assisted living residents, or inpatients with confirmed diagnosis of symptomatic COVID-19 who are age 60+ or age 45+ with known drug-treated diabetes or hypertension, obesity (BMI>30 kg/m2), or established cardiovascular disease. Exclusions: ventilated patients (due to additional complexities with enteral delivery and less plausible mechanisms for benefits in these late stages), pregnancy, and asymptomatic cases. For experiment 2, the population will be those at high risk of contracting the virus such as health care workers, first responders, and nursing home/assisted living caregivers.

Treatment: Active treatment: oral zinc (60 mg/d)+quercetin (500 mg bid)+Vitamin E (200 mg/d)+EGCG (500 mg/d)×3 weeks. Active treatment will likely be provided as separate agents, given potential for time delays and increased costs of reformulating into a single agent. Control: placebo agents.

The active agents were selected based on evidence for their combined ability to enhance anti-viral immunity and also reduce excessive inflammatory responses, as well as their safety. Such nutrient compounds will be tested both singly and in various combinations. The proposed combination treatment is particularly attractive given the complementary mechanistic pathways of these different compounds: e.g., zinc inhibiting viral RNA polymerase and replication; zinc and vitamin E jointly increasing T cell function and reducing inflammation; quercetin blocking viral entry and mitigating excessive inflammatory responses and lung damage; and EGCG exhibiting anti-pathogen and anti-inflammatory effects. Several different combinations will be tested. If clinical benefits are seen with a combination, as expected, then future studies will disentangle which specific nutrient(s) are most responsible for the observed benefit. In parallel, the combination therapy can be rapidly translated into practice to provide timely benefits to a world in crisis.

Subjects will be given the combination treatment for a period of at least three weeks and progression of disease will be monitored routinely (daily). Risk of clinical progression will be determined as rate of hospitalization, rate of mechanical ventilation, and rate of death for inpatients. For outpatients, final disease outcome, need for hospitalization, need for oxygen supplementation, and self-reported symptoms will be measured. For the second trial regarding prevention of infection, the end-points measured will include rate of infection and severity of any subsequent disease. We expect that treatment with this combination of compounds will (1) synergistically improve the clinical outcome of infected subjects and (2) reduce transmission of SARS-CoV-2. For those treated individuals who become infected, we expect the severity of their disease will be less than that of a comparable placebo control group.

### Example 2

Design: Proof-of-concept randomized, placebo-controlled trial.

Specific Aim: A first trial will test a combination of 4 natural compounds for efficacy in reducing severity of clinical symptoms in patients infected with COVID-19. A second trial will test the ability of the disclosed combinations to improve objective measures of immune system function in uninfected adults

Population: Higher risk outpatients (including nursing home/assisted living residents) age 70+ or age 50+ with at least one major risk factor (drug-treated diabetes, drug-treated hypertension, severe obesity (BMI>40 kg/m2), or history of prior heart attack or stroke).
Treatment Arm: patients who have a confirmed diagnosis of COVID-19.
Prevention Arm: patients who are not known to be infected with COVID-19.

(Note: Some pharmacologic agents are being tested in proof-of-concept studies in critically ill, ventilated patients. Given the mechanisms of action of the natural compounds being tested in the present trial, clinical benefits are more likely to be seen in the initial pathogen response as well as for reductions in the severity of disease progression, and not in late stage, critically ill patients when pharmacologic immunosuppression may be more promising.).

Treatment: Active treatment: Oral zinc (60 mg/d)+quercetin (500 mg bid)+Vitamin E (200 mg/d)+EGCG (500 mg/d) provided for:
Treatment Arm: 3 weeks.
Prevention Arm: 3 months.

Control: placebo agents.

Primary Endpoints:
Treatment Arm: Clinical symptoms, including peak severity of symptoms (primary) and duration (days) of symptoms (secondary), assessed by validated questionnaire.
Prevention Arm: Antibody response to influenza vaccine (primary) and delayed-type hypersensitivity skin response (DTH) (secondary).

Secondary endpoints: In subsets of subjects, mechanistic studies will include assessment of blood levels of zinc, vitamin E, EGCG and quercetin, key inflammatory cytokines, measures of endothelial function such as ICAM-1 and VCAM-1, and measures of T cell mediated function. In addition, while currently accurate methods to access COVID-19 viral load are not yet available, in discussion with colleagues at NIH, a test is being developed and they will make it accessible to us when available. Among patients infected with COVID-19, we will also assess progression to hospitalization, progression to mechanical ventilation, and all-cause mortality.

Sample Size and Power Calculation:
Treatment Arm: A total of 200 patients (100 randomized to active treatment, 100 to placebo) treated for 3 weeks each
Prevention Arm: A total of 120 patients (60 randomized to active treatment, 60 to placebo) treated for 3 months each

Statistical analyses will be based on separate hypotheses and separate two-tailed alpha=0.05 for the treatment arm and the prevention arm. The sample size in the treatment arm provides 80% power to detect a 20% reduction in symptom severity, assuming a 10% drop-out rate; and based on mean (SD) symptom score of 10 (4) in the control group. The sample size in the prevention arm is based on prior trial demonstrating efficacy of nutrient interventions for improving vaccine responsiveness, assuming 20% drop-out.

Trial implementation and data collection: We understand that rapid and accurate data collection is critical, while maintaining low site and subject burden, subject safety, and team member safety. We will utilize several approaches for efficient and valid electronic Clinical Outcome Assessments (eCOA), remote data monitoring, real-time data insights, safety monitoring, and accelerated time-to-data-lock for this pandemic setting. These approaches include:

Virtual Trials and Telemedicine: These offer optimized approaches to ensure patient safety, data quality and continuity of study delivery to execute a clinical trial in challenging times. The team includes individuals who help with consent, medical records, scheduling virtual visits, IMP accountability, and managing patient referral workflow.

Home Health Nursing, Direct-to-Patient Shipment and Connected Devices to help patients receive the treatment, assessments, tests and follow-up support needed. This will include virtual patient visits and support teams, nurse and phlebotomist home visits as needed, support getting to sites or local labs, devices to collect vital signs and other important patient safety data from home, and electronic patient reported outcomes (ePRO, such as eDiaries).

Remote Monitoring: We will utilize remote data monitoring to address site access restrictions, reduced site staff and professional clinical research associate (CRA) mobility issues, and to protect data quality, as permitted by local regulations. With remote monitoring, investigator meetings and site initiation visits can be virtual, recorded and placed on the trial portal for future reference; CRAs can log in and monitor data and source data remotely (including eConsent); CRAs can work with site staff via skype/phone on query resolution; and ongoing data review is conducted by a central monitor in a risk-based monitoring model. Remote monitoring activities will include checking patient visits, treatment distribution and compliance, protocol deviations, lab reports, etc.

Summary: This proof-of-concept trial will rapidly evaluate a highly promising combination of natural compounds for efficacy for treating COVID-19 and for boosting the immune response (relevant to both prevention and vaccine responsiveness) in uninfected individuals. Leveraging our collaboration with IQVIA Biotech, the entire trial can be completed within 1 year, providing highly relevant findings in a short amount of time. The compounds have very attractive safety profiles, of crucial importance for identifying a treatment for COVID-19 and the immune response that can be widely used. And, the findings of this trial, whatever the outcome, will be highly informative and relevant for understanding the effects of these nutrients on immune health. COVID-19 is going to be with us for many years, making this trial highly relevant for millions around the globe.

### REFERENCES

1. Gao Y, Yan L, Huang Y, et al. Structure of the RNA-dependent RNA polymerase from COVID-19 virus. Science. Apr. 10, 2020.
2. to Velthuis A J, van den Worm S H, Sims A C, Baric R S, Snij der E J, van Hemert M J. Zn(2+) inhibits coronavirus and arterivirus RNA polymerase activity in vitro and zinc ionophores block the replication of these viruses in cell culture. PLoS Pathog. Nov. 4, 2010; 6(11):e1001176.
3. Micholas Smith J C S. Repurposing Therapeutics for COVID-19: Supercomputer-Based Docking to the SARS-CoV-2 Viral Spike Protein and Viral Spike Protein-Human ACE2 Interface. ChemRxiv. 2020.
4. Chen L, Li J, Luo C, et al. Binding interaction of quercetin-3-beta-galactoside and its synthetic derivatives with SARS-CoV 3CL(pro): structure-activity relationship studies reveal salient pharmacophore features. Bioorg Med Chem. Dec. 15, 2006; 14(24):8295-8306.
5. Polansky H, Lori G. Coronavirus (COVID-19), First Indication of Efficacy of Gene-Eden-VIR/Novirin in SARS-CoV-2 Infections. Int J Antimicrob Agents. Apr. 10, 2020:105971.
6. Kim Y, Narayanan S, Chang K O. Inhibition of influenza virus replication by plant-derived isoquercetin. Antiviral Res. November 2010; 88(2):227-235.
7. Davis J M, Murphy E A, McClellan J L, Carmichael M D, Gangemi J D. Quercetin reduces susceptibility to influenza infection following stressful exercise. Am J Physiol Regul Integr Comp Physiol. August 2008; 295(2):R505-509.
8. Hsu S. Compounds Derived from Epigallocatechin-3-Gallate (EGCG) as a Novel Approach to the Prevention of Viral Infections. Inflammation & allergy drug targets. 2015; 14(1):13-18.
9. Xu J, Xu Z, Zheng W. A Review of the Antiviral Role of Green Tea Catechins. Molecules (Basel, Switzerland). Aug. 12, 2017; 22(8).
10. Kaihatsu K, Yamabe M, Ebara Y. Antiviral Mechanism of Action of Epigallocatechin-3-O-gallate and Its Fatty Acid Esters. Molecules (Basel, Switzerland). Sep. 27, 2018; 23(10).
11. Wintergerst E S, Maggini S, Hornig D H. Contribution of selected vitamins and trace elements to immune function. Ann Nutr Metab. 2007; 51(4):301-323.
12. Wu D, Lewis E D, Pae M, Meydani S N. Nutritional Modulation of Immune Function: Analysis of Evidence, Mechanisms, and Clinical Relevance. Frontiers in immunology. 2018; 9:3160.
13. Meydani S N, Barnett J B, Dallal G E, et al. Serum zinc and pneumonia in nursing home elderly. Am J Clin Nutr. October 2007; 86(4):1167-1173.
14. Falsey A R, Dallal G E, Formica M A, et al. Long-term care facilities: a cornucopia of viral pathogens. J Am Geriatr Soc. July 2008; 56(7):1281-1285.
15. Bonaventura P, Benedetti G, Albarede F, Miossec P. Zinc and its role in immunity and inflammation. Autoimmun Rev. April 2015; 14(4):277-285.
16. Hayek M G, Taylor S F, Bender B S, et al. Vitamin E supplementation decreases lung virus titers in mice infected with influenza. J Infect Dis. July 1997; 176(1):273-276.
17. Han S N, Meydani M, Wu D, et al. Effect of long-term dietary antioxidant supplementation on influenza virus infection. J Gerontol A Biol Sci Med Sci. October 2000; 55(10):B496-503.
18. Lewis E D, Meydani S N, Wu D. Regulatory role of vitamin E in the immune system and inflammation. IUBMB Life. April 2019; 71(4):487-494.
19. Hennet T, Peterhans E, Stocker R. Alterations in antioxidant defences in lung and liver of mice infected with influenza A virus. J Gen Virol. January 1992; 73 (Pt 1):39-46.
20. Nonnecke B J, McGill J L, Ridpath J F, Sacco R E, Lippolis J D, Reinhardt T A. Acute phase response elicited by experimental bovine diarrhea virus (BVDV) infection is associated with decreased vitamin D and E status of vitamin-replete preruminant calves. Journal of dairy science. September 2014; 97(9):5566-5579.
21. Khomich O A, Kochetkov S N, Bartosch B, Ivanov A V. Redox Biology of Respiratory Viral Infections. Viruses. July 26, 2018; 10(8).
22. Wu C, Chen X, Cai Y, et al. Risk Factors Associated With Acute Respiratory Distress Syndrome and Death in Patients With Coronavirus Disease 2019 Pneumonia in Wuhan, China. JAMA internal medicine. Mar. 13, 2020.
23. Farazuddin M, Mishra R, Jing Y, Srivastava V, Comstock A T, Sajjan U S. Quercetin prevents rhinovirus-induced progression of lung disease in mice with COPD phenotype. PLoS One. 2018; 13(7):e0199612.
24. Ganesan S, Faris A N, Comstock A T, et al. Quercetin inhibits rhinovirus replication in vitro and in vivo. Antiviral Res. June 2012; 94(3):258-271.
25. Kumar P, Sharma S, Khanna M, Raj H G. Effect of Quercetin on lipid peroxidation and changes in lung morphology in experimental influenza virus infection. Int J Exp Pathol. June 2003; 84(3):127-133.
26. Ge M, Xiao Y, Chen H, Luo F, Du G, Zeng F. Multiple antiviral approaches of (−)-epigallocatechin-3-gallate (EGCG) against porcine reproductive and respiratory syndrome virus infection in vitro. Antiviral Res. October 2018; 158:52-62.
27. Liu W, Dong M, Bo L, et al. Epigallocatechin-3-gallate suppresses alveolar epithelial cell apoptosis in seawater aspiration-induced acute lung injury via inhibiting STAT1-caspase-3/p21 associated pathway. Mol Med Rep. January 2016; 13(1):829-836.
28. Liu R, Xu F, Si S, Zhao X, Bi S, Cen Y. Mitochondrial DNA-Induced Inflammatory Responses and Lung Injury in Thermal Injury Rat Model: Protective Effect of Epigallocatechin Gallate. J Burn Care Res. September/October 2017; 38(5):304-311.
29. Zhao X D, Liu H, Li T, Gong Q, Zhang W L. Epigallocatechin Gallate Attenuates Hip Fracture-Induced Acute Lung Injury by Limiting Mitochondrial DNA (mtDNA) Release. Medical science monitor: international medical journal of experimental and clinical research. Jul. 12, 2017; 23:3367-3372.
30. Shen H, Wu N, Liu Z, Zhao H, Zhao M. Epigallocatechin-3-gallate alleviates paraquat-induced acute lung injury and inhibits upregulation of toll-like receptors. Life Sci. Feb. 1, 2017; 170:25-32.
31. Xu M J, Liu B J, Wang C L, et al. Epigallocatechin-3-gallate inhibits TLR4 signaling through the 67-kDa laminin receptor and effectively alleviates acute lung injury induced by H9N2 swine influenza virus. Int Immunopharmacol. November 2017; 52:24-33.
32. Ling J X, Wei F, Li N, et al. Amelioration of influenza virus-induced reactive oxygen species formation by epigallocatechin gallate derived from green tea. Acta Pharmacol Sin. December 2012; 33(12):1533-1541.
33. Lee I T, Lin C C, Lee C Y, Hsieh P W, Yang C M. Protective effects of (−)-epigallocatechin-3-gallate against TNF-alpha-induced lung inflammation via ROS-dependent ICAM-1 inhibition. The Journal of nutritional biochemistry. January 2013; 24(1):124-136.
34. Nguyen T T, Woo H J, Kang H K, et al. Flavonoid-mediated inhibition of SARS coronavirus 3C-like protease expressed in Pichia pastoris. Biotechnol Lett. May 2012; 34(5):831-838.
35. Zhang L, Liu Y. Potential interventions for novel coronavirus in China: A systematic review. Journal of medical virology. May 2020; 92(5):479-490.
36. Science M, Johnstone J, Roth D E, Guyatt G, Loeb M. Zinc for the treatment of the common cold: a systematic review and meta-analysis of randomized controlled trials. Cmaj. Jul. 10, 2012; 184(10):E551-561.
37. Prasad A S, Beck F W, Bao B, et al. Zinc supplementation decreases incidence of infections in the elderly: effect of zinc on generation of cytokines and oxidative stress. Am J Clin Nutr. March 2007; 85(3):837-844.
38. Meydani S N, Leka L S, Fine B C, et al. Vitamin E and respiratory tract infections in elderly nursing home residents: a randomized controlled trial. JAMA. Aug. 18, 2004; 292(7):828-836.
39. Heinz S A, Henson D A, Austin M D, Jin F, Nieman D C. Quercetin supplementation and upper respiratory tract infection: A randomized community clinical trial. Pharmacol Res. September 2010; 62(3):237-242.
40. Furushima D, Ide K, Yamada H. Effect of Tea Catechins on Influenza Infection and the Common Cold with a Focus on Epidemiological/Clinical Studies. Molecules (Basel, Switzerland). Jul. 20, 2018; 23(7).
41. Nieman D C, Henson D A, Gross S J, et al. Quercetin reduces illness but not immune perturbations after intensive exercise. Med Sci Sports Exerc. September 2007; 39(9):1561-1569.
42. Bou Ghanem E N, Clark S, Du X, et al. The alpha-tocopherol form of vitamin E reverses age-associated susceptibility to Streptococcus pneumoniae lung infection by modulating pulmonary neutrophil recruitment. J Immunol. Feb. 1, 2015; 194(3):1090-1099.
43. Belisle S E, Hamer D H, Leka L S, et al. IL-2 and IL-10 gene polymorphisms are associated with respiratory tract infection and may modulate the effect of vitamin E on lower respiratory tract infections in elderly nursing home residents. Am J Clin Nutr. July 2010; 92(1):106-114.

1. A composition comprising at least two components selected from the group consisting of zinc, quercetin, vitamin E, and epigallocatechin gallate (EGCG).

2. The composition of claim 1, wherein the composition comprises at least three of or all four of zinc, quercetin, vitamin E, and EGCG.

3. The composition of claim 1, wherein the composition provided as a food product or nutritional supplement.

4. The composition of claim 1, wherein the composition comprises at least two of:
a) 10-75 mg of zinc;
b) 100-1500 mg of quercetinl
c) 50-800 mg of vitamin E; or
d) 300-600 my of EGCG.

5-7. (canceled)

8. The composition of claim 1, wherein the composition is formulated into a dosage unit for daily administration.

9. A dose pack comprising a multiplicity of doses of at least two components selected from the group consisting of zinc, quercetin, vitamin E, and epigallocatechin gallate (EGCG), wherein the dose pack is configured to isolate doses of the at least two components.

10. The dose pack of claim 9, wherein at least three or all four of the components are included in the multiplicity of doses.

11. (canceled)

12. The dose pack of claim 9, wherein the at least two components are comprised in a unitary dosage form or in at least two dosage forms in each dose.

13. (canceled)

14. The dose pack of claim 9, wherein each dose is for daily administration.

15. The dose pack of claim 9, wherein the dose comprises at least two of:
a) 10-75 mg of zinc;
b) 100-1500 mg quercetin;
c) 50-800 mg Vitamin E; or
d) 300-600 mg of EGCG.

16-18. (canceled)

19. A method of administering the dose of the dose pack of claim 9 to a subject.

20. The method of claim 19, wherein the subject has tested positive for COVID-19/SARS-CoV-2 or has been exposed to COVID-19/SARS-CoV-2.

21. (canceled)

22. A method of administering to a subject at least three components selected from the group consisting of zinc, quercetin, vitamin E, and EGCG; wherein the method inhibits viral infection or replication in the subject.

23. (canceled)

24. The method of claim 22, wherein the viral infection is COVID-19/SARS-CoV-2.

25. The method of claim 22, wherein the subject has been exposed to COVID-19/SARS-CoV-2, is at high risk for COVID-19/SARS-CoV-2, or has tested positive for COVID-19/SARS-CoV-2.

26. (canceled)

27. A method of treating a subject for COVID-19/SARS-CoV-2, the method comprising
(a) selecting a subject who has tested positive for COVID-19/SARS-CoV-2; and
(b) administering to the subject the dose pack of claim 9.

28. The method of claim 27, wherein the method:
a) reduces the hospitalization time, intubation time, and/or disease duration of the subject;
b) curtails the clinical progression and/or reduces the symptom severity of COVID-19; or
c) both (a) and (b).

29. (canceled)

30. A method of increasing the immune response to a vaccine for COVID-19/SARS-CoV-2, the method comprising:
(a) selecting a subject who will be receiving a vaccine for COVID-19/SARS-CoV-2;
(b) administering to the subject the dose pack of claim 9; and
(c) administering the vaccine for COVID-19/SARS-CoV-2.

31. The method of claim 30, wherein the subject may be at risk for an attenuated immune response to the COVID-19/SARS-CoV-2 vaccine.

32. The method of claim 30, wherein the dose pack is administered at least daily for at least one week or for at least two weeks prior to the vaccine administration.

33. (canceled)

34. The method of claim 30, wherein the dose pack comprises at least three components or all four components selected from the group consisting of zinc, quercetin, vitamin E, and EGCG.

35. (canceled)

36. The method of claim 22, wherein the at three components are selected from:
a) zinc administered to the subject at 10-75 mg/d;
b) quercetin administered to the subject at 100-1500 mg/d;
c) vitamin E administered to the subject at 50-800 mg/d; and
d) EGCG administered to the subject at 300-600 mg/d.

37-40. (canceled)

41. The method of claim 22, wherein the subject is considered to be at high risk of severe illness from COVID-19.

42. The method of claim 22, wherein the method:
a) inhibits viral entry into cells or viral reproduction in the subject;
b) increases T cell function in the subject;
c) reduces inflammation, cytokine levels, and/or reactive oxygen species in the subject;
d) reduces tissue damage in the subject; or
e) any combination of (a)-(d).

43-45. (canceled)
